(19) United States
(12) Patent Application Publication
(10) Pub. No.: US 2024/0277533 A1
(43) Pub. Date: Aug. 22, 2024

(54) METHOD FOR USING A DISPOSABLE PENILE SHEATH

(71) Applicant: Sonny Willis, Manor, TX (US)

(72) Inventor: Sonny Willis, Manor, TX (US)

(21) Appl. No.: 18/112,586

(22) Filed: Feb. 22, 2023

(51) Int. Cl. A61F 13/471

(52) U.S. Cl. CPC A61F 13/471; A61F 2013/15121

(57) ABSTRACT

A method for using a disposable penile sheath for containing urinary incontinence after voluntary urination includes the steps of urinating voluntarily through a penis and providing a receptacle thereafter. The penis inserts through an opening into the receptacle wherein the receptacle contains urinary incontinence after voluntary urination. The penis is removed from the opening of the receptacle and the receptacle is disposed of thereafter.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sheath and more particularly pertains to a new sheath for containing urinary incontinence after voluntary urination.

### (2) Description of Related Art Including Information Disclosed Under 37 CFR 1.97 and 1.98

The prior art relates to sheaths and includes a variety of sheaths enwrapping a penis to contain urine. Known prior art does not include a sheath enwrapping a penis after voluntary urination to contain urinary incontinence and disposing of the sheath thereafter.

### BRIEF SUMMARY OF THE INVENTION

An embodiment of the disclosure meets the needs presented above by generally comprising a method for using a disposable penile sheath including the steps of urinating voluntarily through a penis and providing a receptacle thereafter. The penis inserts through an opening into the receptacle wherein the receptacle contains urinary incontinence after voluntary urination. The penis is removed from the opening of the receptacle and the receptacle is disposed of thereafter.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a method for using a disposable penile sheath according to an embodiment of the disclosure.

FIG. 2 is a front view of an alternative embodiment of the disclosure.

FIG. 3 is a top isometric view of an embodiment of the disclosure.

FIG. 4 is a top view of an alternative embodiment of the disclosure.

FIG. 5 is a cross-sectional view of an alternative embodiment of the disclosure taken along Line 5-5 of FIG. 2.

FIG. 6 is an in-use view of an embodiment of the disclosure.

### DETAILED DESCRIPTION OF THE INVENTION

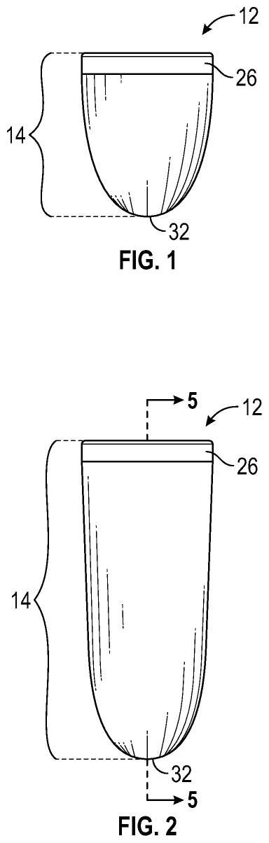

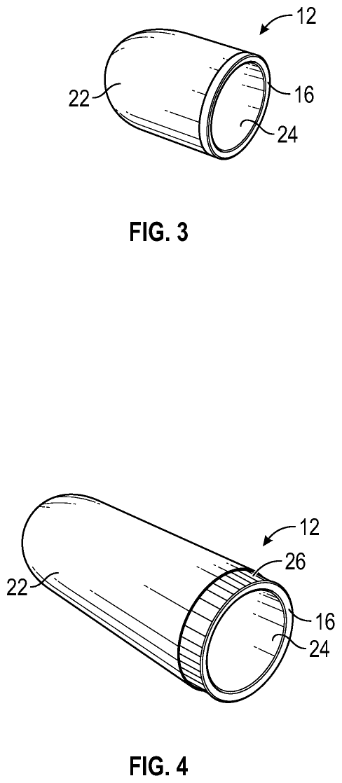

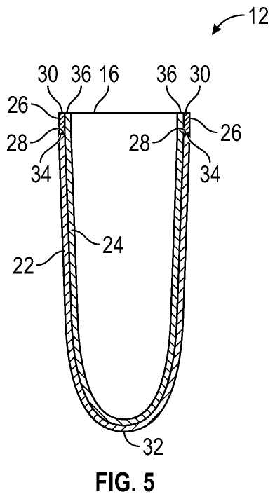

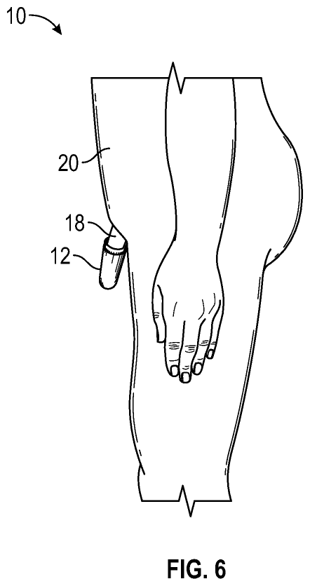

With reference now to the drawings, and in particular to FIGS. 1 through 6 thereof, a new sheath embodying the principles and concepts of an embodiment of the disclosure and generally designated by the reference numeral 10 will be described.

As best illustrated in FIGS. 1 through 6, the method for using a disposable penile sheath 10 generally comprises a receptacle 12. The receptacle 12 is elongated for a length 14 and has an opening 16 therein. The length 14 may be from 2.00 inches to 6.00 inches such that the length 14 may cover a tip of a penis 18 of a user 20 or the length 14 may cover a majority of the penis 18.

The receptacle 12 includes an exterior layer 22 and an interior layer 24. The exterior layer 22 comprises a liquid impermeable material such as rubber to facilitate containing urine within the receptacle 12. The interior layer 24 comprises an absorbent material and normally comprises a tissue paper being soft on the skin of the user 20. An elastic band 26 may be coupled to the receptacle 12 and extend around the opening 16. The elastic band 26 has a proximal edge 28 and distal edge 30 relative to a closed end 32 of the receptacle 12. The proximal edge 28 is coupled to a peripheral edge 34 of the exterior layer 22 and the distal edge 30 is coupled to a perimeter edge 36 of interior layer 24. The elastic band 26 typically comprises an elastomeric material being compressible against the penis 18 wherein the elastic band 26 facilitates securing the receptacle 12 to the penis 18.

In use, the user 20 urinates voluntarily through the penis 18 and provides the receptacle 12 thereafter. The penis 18 inserts through the opening 16 into the receptacle 12 wherein the receptacle 12 contains urinary incontinence after voluntary urination. The penis 18 may be removed from the opening 16 of the receptacle 12 and the receptacle 12 may be disposed of thereafter.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A method for using a disposable penile sheath configured to contain urinary incontinence after voluntary urination comprising the steps of:
urinating voluntarily through a penis;
providing a receptacle;
inserting said penis into said receptacle wherein said receptacle contains urinary incontinence after voluntary urination;
removing said penis from said opening of said receptacle; and
disposing of said receptacle.

2. The method for using a disposable penile sheath of claim 1, wherein the step of providing a receptacle further includes said receptacle being elongated and having an opening therein, said receptacle including an exterior layer and an interior layer.

3. The method for using a disposable penile sheath of claim 2, wherein the step of providing a receptacle further includes said exterior layer comprising a liquid impermeable material.

4. The method for using a disposable penile sheath of claim 2, wherein the step of providing a receptacle further includes said interior layer comprising an absorbent material.

5. The method for using a disposable penile sheath of claim 4, wherein the step of providing a receptacle further includes said interior layer comprising a tissue paper.

6. The method for using a disposable penile sheath of claim 3, wherein the step of providing a receptacle further includes an elastic band being coupled to said receptacle extending around said opening.

7. The method for using a disposable penile sheath of claim 6, wherein the step of providing a receptacle further includes said elastic band having a proximal edge and distal edge relative to a closed end of said receptacle.

8. The method for using a disposable penile sheath of claim 7, wherein the step of providing a receptacle further includes said proximal edge being coupled to a peripheral edge of said exterior layer.

9. The method for using a disposable penile sheath of claim 7, wherein the step of providing a receptacle further includes said distal edge being coupled to a perimeter edge of interior layer.

10. The method for using a disposable penile sheath of claim 6, wherein the step of providing a receptacle further includes said elastic band comprising an elastomeric material.

11. A method for using a disposable penile sheath configured to contain urinary incontinence after voluntary urination comprising the steps of:
urinating voluntarily through a penis;
providing a receptacle, said receptacle being elongated and having an opening therein, said receptacle including an exterior layer and an interior layer, said exterior layer comprising a liquid impermeable material, said interior layer comprising an absorbent material, said interior layer comprising a tissue paper, an elastic band being coupled to said receptacle extending around said opening, said elastic band having a proximal edge and distal edge relative to a closed end of said receptacle, said proximal edge being coupled to a peripheral edge of said exterior layer, said distal edge being coupled to a perimeter edge of interior layer, said elastic band comprising an elastomeric material;
inserting said penis into said receptacle wherein said receptacle contains urinary incontinence after voluntary urination;
removing said penis from said opening of said receptacle; and
disposing of said receptacle.
